(19) United States
(12) Patent Application Publication
(10) Pub. No.: US 2002/0034773 A1
(43) Pub. Date: Mar. 21, 2002

(54) S100 proteins and autoantibodies as serum markers for cancer

(76) Inventors: Samir M. Hanash, Ann Arbor, MI (US); David Misek, Ann Arbor, MI (US); Latha Prasannan, Marshfield, WI (US)

Correspondence Address: BAKER & BOTTS, L.L.P., 30 ROCKEFELLER PLAZA, NEW YORK, NY, 10112, US

(21) Appl. No.: 09/848,948

(22) Filed: May 4, 2001

Related U.S. Application Data
(63) Continuation of application No. PCT/US99/26226, filed on Nov. 5, 1999.
(60) Provisional application No. 60/107,209, filed on Nov. 5, 1998.

(51) Int. Cl. G01N 33/574

(52) U.S. Cl. 435/7.23

(57) ABSTRACT

The present invention relates to screening methods for diagnosis and prognosis of cancer in a subject by means of detecting increased levels of S100 protein in sera and other biological fluids of the subject. The method of the invention can also be used to identify subjects at risk for developing cancer. The method of the invention involves the use of subject derived serum samples, or other biological fluid samples, to determine the occurrence and level of circulating S100 proteins. The invention also provides screening methods for diagnosis, prognosis, or susceptibility to cancer in a subject by means of detecting the presence of serum autoantibodies to specific S100 protein antigens in sera from subjects. The present invention further provides for kits for carrying out the above described screening methods. Such kits will be used to screen patients for increased levels of S100 protein, or for the detection of autoantibodies to S100 proteins, as a diagnostic and prognostic indicator of disease.

### 1. INTRODUCTION

The present invention relates to screening methods for diagnosis and prognosis of cancer in a subject by means of detecting increased levels of S100 protein in sera and other biological fluids of the subject. The method of the invention can also be used to identify subjects at risk for developing cancer. The method of the invention involves the use of subject derived serum samples, or other biological fluid samples, to determine the occurrence and level of specific circulating S100 proteins. The invention also provides screening methods for diagnosis, prognosis, or susceptibility to cancer in a subject by means of detecting the presence of serum autoantibodies to specific S100 protein antigens in sera from subjects. The present invention further provides for kits for carrying out the above described screening methods. Such kits will be used to screen subjects for increased levels of S100 protein, or for the detection of autoantibodies to S100 proteins, as a diagnostic, predictive or prognostic indicator of cancer. The invention is demonstrated by way of example in which increased levels of specific S100 proteins were identified in serum samples derived from subjects with cancer. In addition, elevated levels of circulating autoantibodies reactive against several S100 proteins were detected in the sera of cancer subjects.

### 2. BACKGROUND OF THE INVENTION

A number of cellular proteins have been demonstrated to occur at increased levels in body fluids of subjects with different types of cancer. The increased levels of such proteins in cancer subjects provide diagnostic and prognostic assays for the presence of cancer. For example, elevated serum levels of prostate specific antigen (PSA) is frequently used as an indicator of the presence of prostate cancer in subjects. The occurrence of antibodies to tumor derived proteins as in the case of mutant p53 has also been noted to occur in some cancers. However, there is little knowledge that allows prediction of which proteins in tumors may be antigenic or may occur at increased levels in body fluids.

S100 proteins are low-molecular-weight calcium binding proteins that are believed to play an important role in various cellular processes such as cytoskeletal/membrane interactions, cell division and differentiation. The S100 family of proteins consists of at least 17 family members and the genes encoding this family of proteins have been localized to human chromosomes lq2l. The S100 proteins possess a characteristic EF-hand domain which binds calcium with high affinity. The designation S100 protein refers to the original protein; other S100 proteins are referred to specifically as, for example, S100-A7, S100-A8, etc.

Two specific S100 proteins, S100-A8 and S100-A9, also referred to as myeloid related protein-8 (MRP-8) and myeloid related protein-14(MRP-14), are composed of two distinct EF-hands flanked by hydrophobic regions at either terminus separated by a central hinge region. S100-A8 has been demonstrated to mediate chemotactic activity on macrophages and a peptide encoded by the hinge region (between the two EF-hands) has been shown to specifically mediate this effect. S100-A8 and S100-A9 have both been shown to be secreted from ganulocytes and monocytes, although, it is presently unclear how these proteins are secreted as they do not possess a classical signal peptide. One possibility is that calcium binding may expose a hydrophobic domain which could allow an interaction with the membrane, thereby resulting in secretion of the molecules. Both S100-A8 and S100-A9 homodimerize and heterodimerize with each other, thus forming complexes of various molecular weights. An antibody against the cystic fibrosis antigen (an epitope formed by heterodimerization of S100-A8 and S100-A9) also will react positively against a 14 kDa antigen which has been shown to be S100-A9.

The expression of S100 protein has been evaluated in a variety of disorders. For example, S100 proteins have been evaluated as serum markers for subjects with inflammatory diseases, including ulcerative colitis, Crohn's disease, and as serum markers for subjects with infectious diseases including AIDs and malaria and for subjects with hematological disease. Evidence has accumulated that indicates that some S100 proteins can alter cellular invasion and metastatic spread of cancer. For example, S100 protein is expressed in dendritic cells in human transitional cell carcinoma of the bladder and the invasive potential of these tumors has been found to correlate with the presence of S100 protein expressing cells (Inoue et al., 1993, Virchows Arch A 422: 351-355). Additionally, the expression of S100A4 correlates with the in vitro invasive potential of glioma cells (Merzak, et al., 1994, Neuropathol App. Neurobiol. 20: 614-619).

Expression of S100 proteins has also been evaluated as a serum marker for melanoma (Henze et al. 199, Dermatology 194:208-212; Buer et al., 1997, Brit. J. Cancer 75:1373-1376; Sherbet et al. 1998, Anticancer Research 18:2415-2422). However, S100 proteins have not been previously considered as serum markers for cancer in general, nor that their detectablity in serum might have prognostic or therapeutic significance in cancer. Additionally, there have not been prior findings of auto-antibodies to S100 proteins in cancer.

### 3. SUMMARY OF THE INVENTION

It is an object of the present invention to provide methods for the diagnostic and prognostic evaluation of cancer, for the identification of subjects possessing a predisposition to cancer, and for monitoring patients undergoing treatment of cancer, based on the detection of increased levels of S100 protein expression in biological fluid samples of subjects. The invention provides methods for detecting overexpression of individual S100 proteins and/or overexpression of groups of S100 proteins as a diagnostic or prognostic indicator of cancer.

The invention comprises assays developed to detect the level of S100 proteins in a subject's serum sample. Such assays include immunoassays wherein the S100 proteins are detected by their interaction with anti-S100 specific antibodies. For example, S100 antibodies or fragments of antibodies may be used to quantitatively detect the presence of S100 proteins in a serum sample.

The invention further relates to diagnostic evaluation and prognosis of cancer by detecting autoantibodies to S100 protein antigens in the serum of subjects with cancer or with precancerous lesions. The detection of increased serum levels of autoantibodies to S100 proteins constitutes a novel strategy for screening, diagnosis and prognosis of cancer.

The present invention relates to the use of the S100 protein antigens in immunoassays designed to detect the presence of serum autoantibodies to the S100 protein antigens. Such immunoassays can be utilized for diagnosis and prognosis of cancer. In accordance with the invention, measurement of S100 autoantibody levels in a subject's serum can be used for the early diagnosis of cancer. Moreover, the monitoring of serum autoantibody levels can be used prognostically to stage progression of the disease.

The invention also relates to the use of S100 proteins as antigens to immunize patients suffering from diseases characterized by the expression of the S100 protein antigens. Stimulation of an immunological response to such antigens, is intended to elicit a more effective attack of tumor cells; such as inter alia inhibiting tumor cell growth or facilitating the killing of tumor cells. The identification of autoantibodies to S100 protein antigens associated with particular cancers provides a basis for immunotherapy of the disease.

The invention further provides for pre-packaged diagnostic kits which will be conveniently used in clinical settings, to diagnose patients having cancer or a predisposition to developing cancer. The kits will also be utilized to monitor the efficiency of compounds used for treatment of cancer.

In a specific embodiment of the invention described herein, an increase in the level of specific S100 proteins was detected in serum samples derived from subjects with lung cancer as well as colon cancer. Additionally, a number of S100 family members were shown to be secreted from breast cancer cells indicating that detection of these specific proteins in body fluid samples can be used for diagnosis and prognosis of subjects with breast cancer. In yet another specific embodiment, autoantibodies against specific S100 proteins were detected in the serum of subjects with cancer. The finding that levels of S100 proteins and S100 autoantibodies are increased in serum of cancer subjects provides a basis for development of diagnostic and prognostic methods as well as a means for monitoring the efficacy of various therapeutic treatments for cancer.

### 4. BRIEF DESCRIPTION OF THE DRAWINGS

FIG. 1A-B. Two dimensional electrophoretic gel patterns of normal colon (FIG. 1A) and a colon tumor (FIG. 1B) from the same subject. The location of some of the S100 proteins is identified with arrows. MRP-14 corresponds to S 100-A9 and MRP-8 corresponds to S100-A8.

FIG. 2. Two dimensional patterns of secreted proteins from breast cancer cells identifying the location of S100 proteins.

FIG. 3. Detection of an protein band corresponding to S100-A9 in serum of lung cancer subjects.

FIG. 4A. Western blot of a lung cancer treated with a serum from a subject with lung adenocarcinoma tumor.

FIG. 4B. Western Blot of lung cancer treated with serum from a normal subject.

### 5. DETAILED DESCRIPTION OF THE INVENTION

The present invention achieves a highly desirable objective, namely providing methods for the diagnostic and prognostic evaluation of subjects with cancer and the identification of subjects exhibiting a predisposition to developing cancer. The assays of the invention comprise methods designed to detect increased levels of S100 protein, or the presence of S100 autoantibodies, in serum or other biological fluids of a subject.

Specifically, the invention encompasses a method for diagnosis and prognosis of cancer in a subject comprising:

(a) detecting at least one S100 protein selected from the group consisting of S100-AG, S100-A7, S100-A8 and S100-A9 in a biological fluid sample derived from a subject; and

(b) comparing the level of protein detected in the subject's sample to the level of protein detected in a control sample, wherein an increase in the level of S100 protein detected in the subject's sample as compared to control samples is an indicator of a subject with cancer or at increased risk for cancer.

A wide variety of protein mixtures that may contain S100 proteins can be prepared or assayed for the level of protein expression. In a preferred embodiment sera and other biological fluids in which secreted proteins localize can be used to screen for increased levels of protein expression.

The present invention encompasses a method for diagnosis and prognosis of a subject with cancer, comprising:

(a) contacting a serum sample derived from a subject with a sample containing S100 protein antigens under conditions such that a specific antigen-antibody binding can occur; and

(b) detecting the presence of immunospecific binding of autoantibodies present in the subject's serum sample to the S100 protein, wherein the presence of immunospecific binding of autoantibodies indicates the presence of cancer.

In a specific embodiment of invention, any member of the S100 protein family can be purified and utilized to screen a subject's serum for the presence of circulating autoantibodies to such protein antigens, by means of sensitive and rapid immunoadsorbent assays or by other procedures.

The present invention also provides for kits for carrying out the above-described methods. The methods can be performed, for example, by utilizing pre-packaged diagnostic kits comprising at least a reagent for detecting S100 protein such as an anti-S100 antibody. Alternatively, the diagnostic kits will comprise an S100 peptide for detection of S100 autoantibodies in a subject derived sample.

The present invention is based on the discovery that levels of S100 proteins in serum are increased in subjects with cancer such as lung cancer or colon cancer. In addition, breast cancer cells were demonstrated to secrete an S100 protein thus indicating that detection of circulating S100 proteins can be used in methods for diagnostic and prognostic evaluation of breast cancer subjects. Additionally, increased levels of circulating autoantibodies reactive against S100 proteins were detected in the serum of cancer subjects.

### 5.1. ASSAYS FOR DETECTION OF S100 EXPRESSION

In accordance with the invention, measurement of levels of S100 proteins in serum or body fluids can be used for the early diagnosis of diseases such as cancer. Moreover, the monitoring of S100 protein levels can be used prognostically to stage the progression of the disease and to evaluate the efficacy of compounds used to treat a cancer subject.

The detection of S100 proteins in a body fluid from a subject can be accomplished by any of a number of methods. Preferred diagnostic methods for the detection of S100 proteins in the serum of a patient can involve, for example, immunoassays wherein S100 proteins are detected by their interaction with an S100 specific antibody. Antibodies useful in the present invention can be used to quantitatively or qualitatively detect the presence of S100 peptides. In addition, reagents other than antibodies, such as, for example, polypeptides that bind specifically to S100 proteins can be used in assays to detect the level of S100 protein expression.

Immunoassays to be used in the practice of the invention include but are not limited to assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement fixation assays, immunoradiometric assays, fluorescent immunoassays, protein A immunoassays, to name but a few.

A biological sample which may contain S100 proteins, such as serum or other biological fluids in which secreted proteins can localize, is obtained from a subject suspected of having a particular cancer or risk for cancer. Immunoassays for detecting expression of S100 protein typically comprise contacting the biological sample, such as a serum sample derived from a subject, with an anti-S100 antibody under conditions such that specific antigen-antibody binding can occur, and detecting or measuring the amount of any immunospecific binding by the antibody. In a specific aspect, such binding of antibody, for example, can be used to detect the presence and increased expression of S100 proteins wherein the detection of increased expression of S100 proteins is an indication of a diseased condition. The levels of S100 protein in a serum sample are compared to norms established for normal individuals and for subjects with a variety of non-cancerous or pre-cancerous disease states.

In an embodiment of the invention, the biological sample, such as a serum sample is brought in contact with a solid phase support or carrier, such as nitrocellulose, for the purpose of immobilizing any proteins present in the sample. The support is then washed with suitable buffers followed by treatment with detectably labeled S100 specific antibody. The solid phase support is then washed with the buffer a second time to remove unbound antibody. The amount of bound antibody on the solid support is then determined according to well known methods. Those skilled in the art will be able to determine optional assay conditions for each determination by employing routine experimentation.

One of the ways in which S100 antibodies can be detectably labeled is by linking the antibody to an enzyme, such as for use in an enzyme immunoassay (EIA) (Voller, A., “The Enzyme Linked Immunosorbent Assay (ELISA)”, 1978, Diagnostic Horizons 2:1-7, Microbiological Associates Quarterly Publication, Walkersville, MD; Voller, A., et al., 1978, J. Clin. Pathol. 31:507-520; Butler, J.E., 1981, Meth. Enzymol. 73:482-523). The enzyme which is bound to the antibody will react with an appropriate substrate, preferably a chromogenic substrate, in such a manner as to produce a chemical moiety that can be detected, for example, by spectrophotometric, fluorimetric, or by visual means. Enzymes that can be used to detectable label the antibody include, but are not limited to, horseradish peroxidase and alkaline phosphatase. The detection can also be accomplished by calorimetric methods that employ a chromogenic substrate for the enzyme.

Detection of S100 antibodies may also be accomplished using a variety of other methods. For example, by radioactively labeling the antibodies or antibody fragments, it is possible to detect S100 protein expression through the use of a radioimmunoassay (RIA) (see, for example, Weintraub, B., Principles of Radioimmunoassays, Seventh Training Course on Radioligand Assay Techniques, The Endocrine Society, March 1986). The radioactive isotope can be detected by such means as the use of a gamma counter or a scintillation counter or by autoradiography.

The antibody may also be labeled with a fluorescent compound. Among the most commonly used fluorescent labeling compounds are fluorescein isothiocyanate, rhodamine, phycoerythrin and fluorescamine. Likewise, a bioluminescent compound may be used to label the SI 00 antibody. The presence of a bioluminescence protein is determined by detecting the presence of luminescence. Important bioluminescence compounds for purposes of labeling are luciferin, luciferase and aequorin.

In a specific embodiment of the invention, expression levels of S100 proteins in biological samples can be analyzed by two-dimensional gel electrophoresis. Methods of two-dimensional electrophoresis are known to those skilled in the art. Biological samples, such as serum samples, are loaded onto electrophoretic gels for isoelectric focusing separation in the first dimension which separates proteins based on charge. A number of first-dimension gel preparations may be utilized including tube gels for carrier ampholytes-based separations or gels strips for immobilized gradients based separations. After first-dimension separation, proteins are transferred onto the second dimension gel, following an equilibration procedure and separated using SDS PAGE which separates the proteins based on molecular weight. When comparing serum samples derived from different subjects, multiple gels are prepared from individual serum samples.

Following separation, the proteins are transferred from the two-dimensional gels onto membranes commonly used for Western blotting. The techniques of Western blotting and subsequent visualization of proteins are also well known in the art (Sambrook et al, “Molecular Cloning, A Laboratory Manual”, 2^{nd} Edition, Volume 3, 1989, Cold Spring Harbor). The standard procedures may be used, or the procedures may be modified as known in the art for identification of proteins of particular types, such as highly basic or acidic, or lipid soluble, etc. (See for example, Ausubel, et al., 1989, Current Protocols in Molecular Biology, Green Publishing Associates and Wiley Interscience, N.Y.). Antibodies that bind to the S100 proteins are utilized in an incubation step, as in the procedure of Western blot analysis. A second antibody specific for the first antibody is utilized in the procedure of Western blot analysis to visualize proteins that reacted with the first antibody.

The detection of S100 protein expression can also be used to monitor the efficacy of potential anti-cancer compounds during treatment. For example, the level of S100 protein expression can be determined before and during treatment. The efficacy of the compound can be followed by comparing S100 expression throughout the treatment. Compounds exhibiting efficacy are those which decrease the level of S100 protein expression as treatment with the compound progresses.

The present invention is demonstrated by way of example wherein elevated levels of an S100 protein was detected in serum samples derived from cancer subjects. In particular, increased levels of S100-A9 were detected in serum samples derived from colon and lung patients. In addition, S100-A7 and S100-A8 proteins were shown to be secreted by breast cancer cells, which provide the basis for diagnostic and prognostic assays for breast cancer. The detection and/or quantitative measurement of S100 proteins in serum or other body fluids can be used in screening of subjects who are at risk for developing certain types of cancers or other proliferative disorders in which the S100 proteins are over expressed. In addition, qualitative differences in the pattern of occurrence in serum or biological fluids of different members of the S100 family of proteins can be used as a screening, diagnostic or prognostic indicator of cancer or cancer risk.

### 5.2. ASSAYS FOR DETECTION OF ANTI-S100 AUTOANTIBODIES

The present invention provides diagnostic and prognostic methods for diseases such as cancer based on detection of circulating S100 autoantibodies in a subject. The method is validated by the use of a biological sample from a subject with cancer and from controls, without cancer. A biological sample which may contain autoantibodies, such as serum, is obtained from a subject suspected of having a particular cancer or suspected of being predisposed to developing cancer. A similar body fluid is obtained from a control subject that does not have cancer.

In accordance with the invention, measurement of autoantibodies reactive against the S100 protein antigens can be used for the early diagnosis of diseases such as cancer. Moreover, the monitoring of autoantibody levels can be used prognostically to stage the progression of the disease. The detection of autoantibodies in a serum sample from a patient can be accomplished by any of a number of methods. Such methods include immunoassays which include, but are not limited to, assay systems using techniques such as Western blots, radioimmuunoassays, ELISA (enzyme linked immunosorbent assay), “sandwich” immunoassays, immunoprecipitation assays, precipitin reactions, gel diffusion precipitin reactions, immunodiffusion assays, agglutination assays, complement fixation assays, immunoradiometric assays, fluorescent immunoassays, protein A immunoassays, to name but a few.

Such an immunoassay is carried out by a method comprising contacting a serum sample derived from a subject with a sample containing the S100 protein antigens under conditions such that specific antigen-antibody binding can occur, and detecting or measuring the amount of any immunospecific binding by the autoantibody. In a specific aspect, such binding of autoantibody by tissue sections, for example, can be used to detect the presence of autoantibody wherein the detection of autoantibody is an indication of a diseased condition. The levels of autoantibodies in a serum sample are compared to the levels present in an analogous serum sample from a subject not having the disorder.

The immunoassays can be conducted in a variety of ways. For example, one method to conduct such assays involves anchoring of S100 protein onto a solid support and detecting anti-S100 antibodies specifically bound thereto. The S100 proteins to be utilized in the assays of the invention can be prepared via recombinant DNA techniques well known in the art. For example, in instances where the nucleotide sequence of a DNA encoding an S100 protein is available, the DNA can be genetically engineered into an appropriate expression vector for large scale preparation of S100 protein. It may be advantageous to engineer fusion proteins that can facilitate labeling, immobilization or detection of the S100 protein. See, for example, the techniques described in Sambrook et al., 1989, Molecular Cloning: A laboratory Manual, Cold Spring Harbor Press, Cold Spring Harbor, N. Y. Alternatively, the S100 protein may be purified from natural sources, e.g., purified from cells, using protein separation techniques well known in the art. Such purification techniques may include, but are not limited to molecular sieve chromatography and/or ion exchange chromatography. In practice, microtitre plates are conveniently utilized as the solid support for the S100 proteins. The surfaces may be prepared in advance and stored.

The present invention is demonstrated by way of example wherein elevated levels of circulating autoantibodies reactive against several S100 protein antigens were detected in the sera of cancer patients. The detection and/or quantitative measurement of circulating anti-S100 autoantibodies in serum can be used in screening of subjects who are at risk for cancer or other proliferative disorders in which S100 proteins are expressed. Additionally, the present invention further relates to the use of the identified protein antigens as immunogens for stimulation of an host immune response against the tumor cells. It is expected that such an approach can be used as a method for inhibiting tumor cell growth or facilitating tumor cell killing in individuals with specific cancers.

### 5.3. IMMUNOTHERAPY

The invention also relates to the use of S100 proteins as antigens to immunize patients suffering from diseases characterized by the expression of the S100 protein antigens. Stimulation of an immunological response to such antigens, is intended to elicit a more effective attack of tumor cells; such as inter alia inhibiting tumor cell growth or facilitating the killing of tumor cells. The identification of autoantibodies to S100 protein antigens associated with particular cancers provides a basis for immunotherapy of the disease.

The patient may be immunized with the S100 protein antigens to elicit an immune response which facilitates killing of tumor cells or inhibiting tumor cell growth. The S100 protein antigens can be prepared using the methods described above for purification of proteins.

In an embodiment of the invention an immunogen comprising a purified S100 protein antigen to which a patient with cancer has developed autoantibodies, is used to elicit an immune response. For administration, the S100 protein antigen should be formulated with a suitable adjuvant in order to enhance the immunological response to the protein antigen. Suitable adjuvants include, but are not limited to mineral gels, e.g. aluminum hydroxide, surface active substances such as lysolecithin, pluronic polyols, polyanions, peptides, oil emulsions, and potentially useful human adjuvants such as BCG (bacilli Calmett-Guerin) and (Corynebacterium parvum). Many methods may be used to introduce the formulations derived above; including but not limited to oral, intradermal, intramuscular, intraperitoneal, intravenous, and subutaneous.

### 5.4. KITS

The present invention further provides for kits for carrying out the above-described assays. The assays described herein can be performed, for example, by utilizing pre-packaged diagnostic kits, comprising at least an S100 peptide (for detection of S100 autoantibodies) or an S100 antibody reagent (for detection of S100 protein), which can be conveniently used, e.g., in clinical settings to diagnose disorders such as cancer.

In a first series of nonlimiting embodiments, a kit according to the invention comprises components for detecting and/or measuring human IgG antibodies directed toward S100 antigen. As one example, where the antibodies are detected and/or measured by enzyme linked immunoabsorbent assay (ELISA), such components may comprise target antigen, in the form of at least one and preferably a plurality of different S100 antigens or epitopes thereof, linked to a solid phase, and a means for detecting a human antibody antibody bound to target antigen. Such means for detection may be, for example, an antibody directed toward the constant region of human IgG (e.g., rabbit anti-human IgG antibody), which may itself be detectably labeled (e.g., with a radioactive, fluorescent, calorimetric or enzyme label), or which may be detected by a labeled secondary antibody (e.g., goat anti-rabbit antibody).

In a second series of nonlimiting embodiments, a kit according to the invention may comprise components which detect and/or measure S100 antigens in the biological sample of a subject. For example, where S100 proteins are detected and/or measured by enzyme linked immunoabsorbent assay (ELISA), such components may comprise an antibody directed to epitopes of the S100 proteins which can be used to detect and/or quantitate the level of S100 expression in the biological sample. The antibody itself may be detectably labeled with a radioactive, flourescent, colorimetric or enzyme label. Alternatively, the kit may contain a labeled secondary antibody.

### 6. EXAMPLE: DETECTION OF A TUMOR ANTIGEN USING SERUM ISOLATED FROM A SUBJECT HAVING CANCER

Subjects with lung and colon cancer were assayed for detection of elevated levels of S100 protein. Tumor tissue as well as normal tissue control was obtained from subjects with the following types of cancer: small cell lung cancer, squamous cell carcinoma of the lung, adenocarcinoma of the lung, esophageal cancer and Barrett's metaplasia, colon cancer, pancreatic cancer, breast cancer, and prostate cancer. An aliquot of whole tissue was solubilized using a solubilization cocktail prior to two-dimensional electrophoresis.

Solubilized proteins were loaded onto electrophoretic gels for separation in the first dimension, based on charge. Two different first dimension gel preparations were utilized namely tube gels for carrier ampholyte based isoelectric focusing (IEF) and gels strips for immobilized pH gradients based separations (IPG). After the first dimension separation, proteins were transferred onto the second dimension gel, following an equilibration procedure and separated in the second dimension based on molecular weight. In some cases multiple gels were prepared from individual serum samples. Proteins patterns were visualized by silver staining and analyzed for differences between normal and tumor tissue and/or for differences between different tumor types by means of a computerized matching process and by means of quantitation of spot intensities.

Many proteins occurred at an increased intensity in particular tumors compared to controls. A set of four proteins occurred at an increased intensity in tumor patterns which were not detectable in controls. The quantitative data in lung cancer for one of the proteins which was later identified as S100-A8 is given in Table I.

TABLE I
Detection of S100-A8 in Tissue From Lung Tumor Patients

| | | | Integrated |
|---|---|---|---|
| Patient | Type | Number | Intensity (Mean) |
| Tumor | Squamous | 9 | 1.84 |
| | Adenocarcinoma | 8 | 1.31 |
| | Small Cell | 9 | 0.34 |
| Normal Tissue | | 26 | Not Detected |

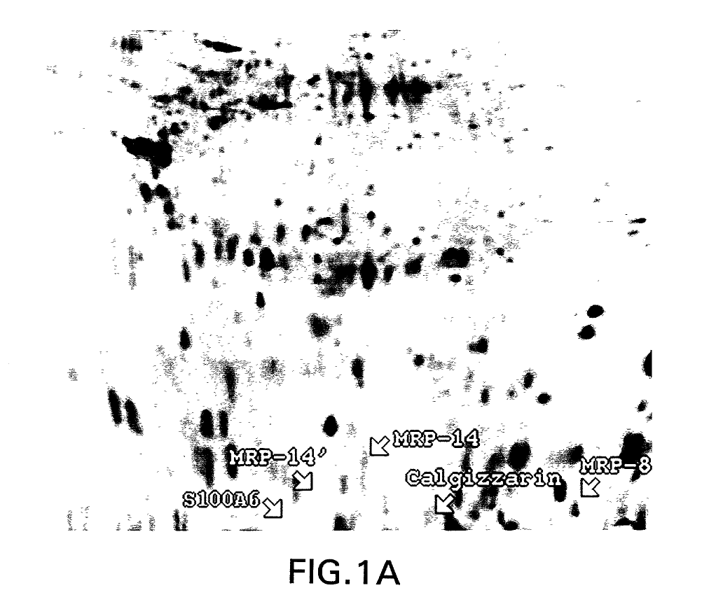

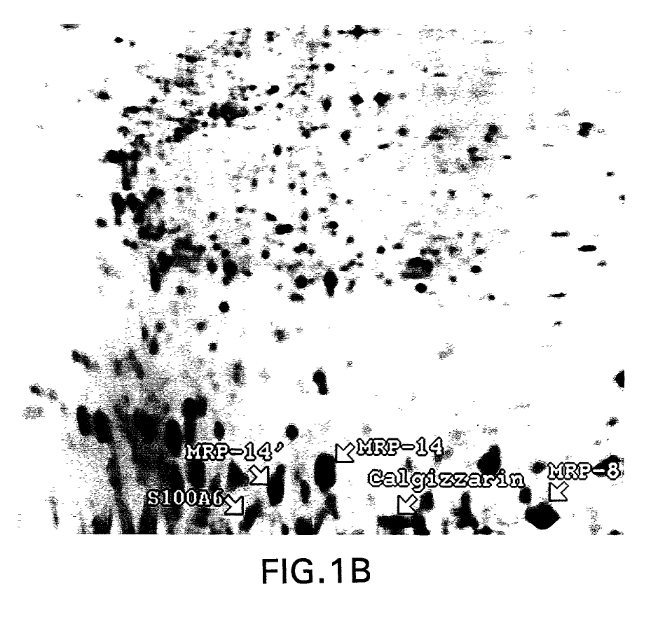

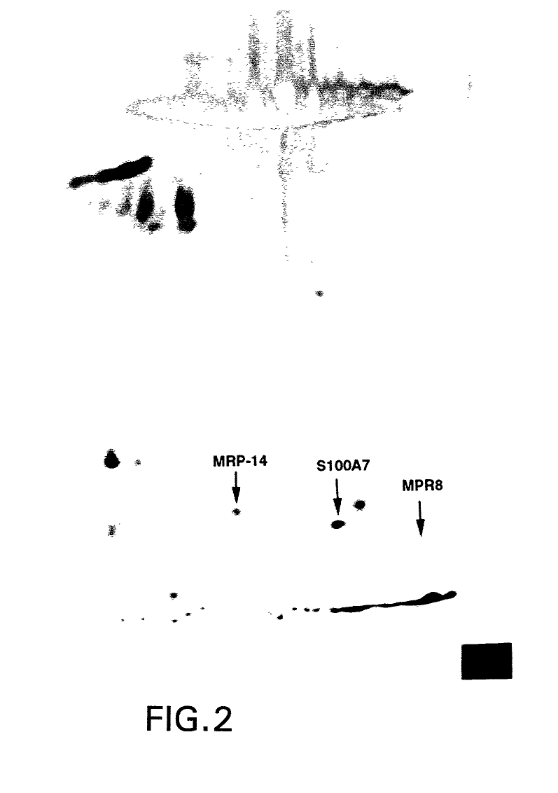

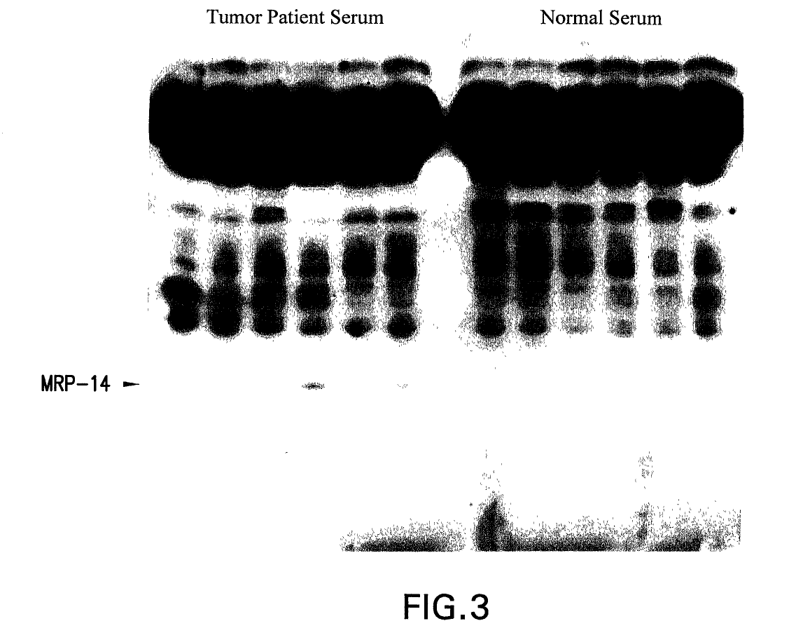

This protein was extracted from 2-D gels and subjected to amino acid sequencing. It gave the N-terminal amino acid sequence MILTELEKALN, which is 100% identical with the reported N-terminal amino acid sequence of the S1 00-A8 protein, found by searching protein databases. The other three proteins in the set were also identified as belonging to the S100 family of proteins. Two were consistent, with S100-A9 which has two translation initiation sites situated 4 amino acids apart, and one with Calgizzerin which is another S100 related protein, as determined by reactivity with monoclonal antibodies and by comparison with published figures of two-dimensional protein separations. The location of these proteins in 2-D patterns of different tumor types is provided in FIGS. 1 and 2.

A commercially available antibody against S100-A9 (DAKO;Carpinteria, Calif.) was utilized for immunohistochemistry and for Western blotting. Immunohistochemistry on sections of tumor tissue and corresponding normal tissue from the same subject with lung cancer was done. The analyzed tissue sections revealed minimal S100-A9 immunoreactivity in the normal lung tissue. The observed increase in reactivity in the tumor tissue was attributed to the presence of infiltrative cells. There was also marked immunoreactivity in the area of normal tissue immediately adjacent to the tumor, resulting from infiltrative cells (i.e., ganulocytes, monocytes and/or macrophage) recruited to the tumor.

An assay was performed to determine whether the S100-A9 antibody would recognize a specific band in the serum of tumor subjects, at levels greater than that which might be present in the serum of normal individuals. Proteins contained in the serum of 14 lung tumor subjects and 14 normal individuals were separated by one dimensional gel electrophoresis, the proteins transferred to PVDF membranes and probed with a commercial antibody (DAKO;Carpinteria, Calif.) that is reactive against the S100-A9 protein. Integrated intensity analysis of reactivity in a band visualized at 14 kDa revealed markedly increased reactivity in the serum from tumor subjects (n=14; mean intensity of 0.46) as compared to that in the serum from normal individuals (n=14; mean intensity of 0.09) (Table II). Another band at 22 kDa was also observed in some tumors and was presumed to result from another immunoreactive form of S100 protein. Similar results were also observed with serum of subjects with colon cancer.

TABLE II
Analysis of MRPl4 Detection in Serum From Lung Tumor Patients

| | | | | Integrated |
|---|---|---|---|---|
| | | | | Intensity |
| Patient | Type | Stage | Number | (Mean) |
| Tumor | All | | 14 | 0.46 |
| | Squamous | I | 4 | 0.65 |
| | Adenocarcinoma | None | 1 | 0.03 |
| | | I | 3 | 0.26 |
| | | IIIA | 2 | 0.42 |
| | Bronchoalveolar | I | 1 | 0.44 |
| | Small Cell | None | 2 | 0.65 |
| | Other (Met. Ovarian) | None | 1 | 0.01 |
| Normal | | | 14 | 0.09 |

A major determinant of the potential of a protein synthesized by tumors to be detected in serum and other biological fluids is its secreted nature. The features of a protein that determine whether it is secreted by cells remains poorly understood. Factors that affect secretory process may depend on the occurrence of post-translational modifications in the protein as well as the activation of certain signaling pathways. In order to facilitate the identification of secretory proteins, a method has been developed to purify proteins secreted by cultured tumor cells, visualize the proteins in two-dimensional gels by coomassie or silver staining and analyze secreted protein spots by N-terminal sequencing or by Mass Spectrometry of their constituent peptides. Using this process three members of the S100 family were identified as secreted proteins in breast cancer. N-terminal sequencing of two secreted proteins identified them as MRP8 and MRP 14 based on the following sequences:

| Amino Acid Sequence | Identify |
|---|---|
| MLTELEKALN | MRP8 |
| MCKMSQECRN | MRP14 |

Additionally, mass spectrometry identified S100A7 as a secreted protein in breast cancer. Seven peptides masses obtained by mass spectrometry matched expected peptide masses for S100A7/These were: 1291.65; 1307.66; 1323.65; 1384.69; 1455.66; 1583.65; and 1711.91.

### 7. EXAMPLE: DETECTION OF AUTOANTIBODIES SPECIFIC FOR S100 PROTEINS IN THE SERA OF SUBJECTS WITH CANCER

Using the method of the present invention, sera from subjects with lung cancer were screened for reactivity against S100 proteins.

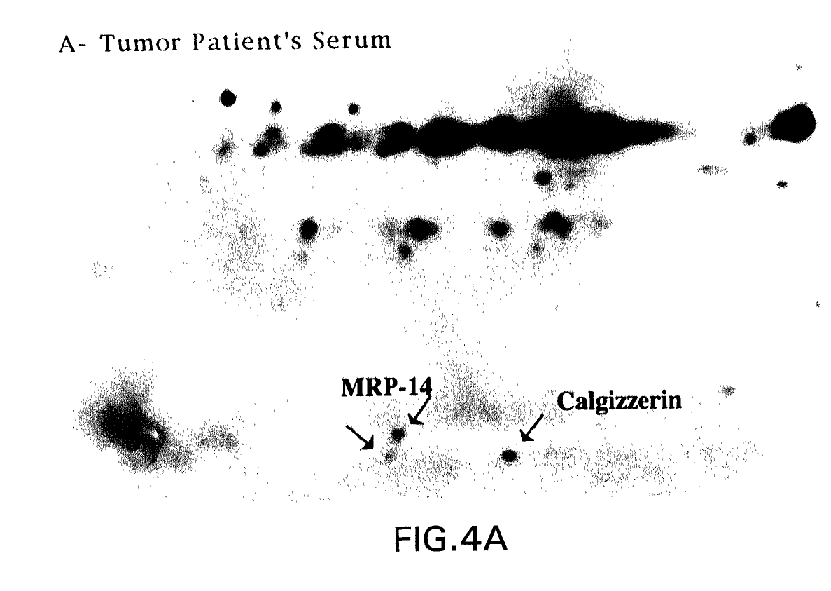

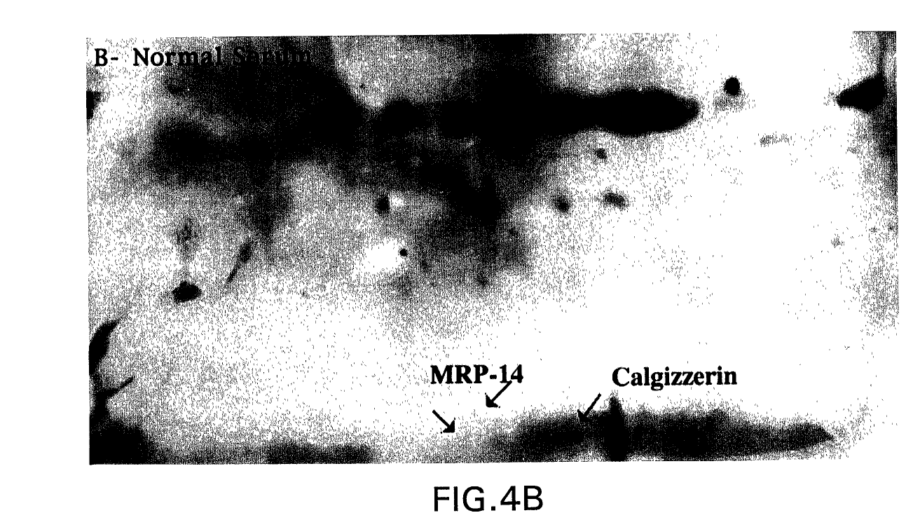

An aliquot of lung tumor tissue was solubilized in a urea cocktail (per liter:8M area, 20 ml Nonidet P-40 surfectant, 20 ml of ampholytes (pH 3.5-10), 20 ml of 2-mercuptoethanol and 0.2 mm of phenylmethylsulfonyl fluoride (PMSF) in distilled demonize H₂O) and 40 micrograms of solubilized protein was loaded onto a carrier ampholyte based (pH 3.8) tube gel and separated in the first dimension for 12,000 volt hours. Following an equilibrium step, the first dimension tube gel was loaded onto a cassette containing the second dimension gel. Electrophoresis in the second dimension was complete when the tracking dye present in the equilibration buffer reached the opposite end of the second dimension gel, in relation to the first dimension gel. Following electrophoresis the separated proteins were transferred onto a polyvinylideme fluoride (PVDF) membrane (millipore). The membrane was preincubated with a blocking buffer and subsequently incubated with serum obtained from a subject with lung adenocarcinoma, which was diluted 1:100 in the buffer solution (Tris-buffered-saline containing 0.01% Between 20 and 1.8 gm/100 ml non-fat dry milk), for 1 hr at room temperature. After three washes with a buffer solution, the membrane was incubated for 1 hr with a horseradish peroxidase conjugated rabbit anti-human antibody (available from Amersham). Reactive proteins were revealed by a chemiluminescent technique. The sera sample from the cancer subject was found to be reactive against a set of S100 proteins identified by microsequencing as S100-A9 and calgizzerin (FIG. 4A-B).

The present invention is not to be limited in scope by the embodiments disclosed in the examples which are intended as an illustration of one aspect of the invention, and any compositions or methods which are functionally equivalent are within the scope of this invention. Indeed, various modifications of the invention in addition to those shown and described herein will become apparent to those skilled in the art from the foregoing description. Such modifications are intended to fall within the scope of the claims. Various publications are cited herein, the contents of which are incorporated, by reference, in their entireties.

1. A method for diagnosis and prognosis of cancer in a subject comprising:
(a) detecting at least one S100 protein selected from the group consisting of S100-AG, S100-A7, S100-A8 and S100-A9 in a biological fluid sample derived from a subject; and
(b) comparing the level of protein detected in the subject's sample to the level of protein detected in a control sample, wherein an increase in the level of S100 protein detected in the subject's sample as compared to a control sample is an indicator of a subject with cancer.

2. The method of claim 1 wherein the S100 protein is detected using an immunoassay.

3. The method of claim 2 wherein the immunoassay is an immunoprecipitation assay.

4. The method of claim 1 wherein the sample is a serum sample.

5. The method of claim 1 wherein the cancer is lung cancer.

6. The method of claim 1 wherein the cancer is breast cancer.

7. The method of claim 1 wherein the cancer is colon cancer.

8. A method for diagnosis of a subject with cancer comprising:
(a) contacting a serum sample derived from a subject with a sample containing S100 protein antigens under conditions such that a specific antigen-antibody binding can occur; and
(b) detecting immunospecific binding of the autoantibodies to the S100 protein in the subject's serum sample, wherein the presence of autoantibodies indicates the presence of cancer.

9. The method of claim 8 wherein the step of detecting the autoantibodies in the subject's serum sample comprises the use of a signal-generating component bound to an antibody that is specific for antibodies in the subject's serum sample.

10. The method of claim 9 wherein the presence of autoantibodies in the serum sample is measured by an immunoassay comprising:
(a) immobilizing one or more S100 protein onto a membrane or substrate;
(b) contacting the membrane or substrate with a subject's serum sample; and
(c) detecting the presence of autoantibodies specific for the S100 protein in the subject's serum sample, wherein the presence of autoantibodies indicates the presence of cancer.

11. The method of claim 8 wherein the cancer is lung cancer.

12. The method of claim 8 wherein the cancer is breast cancer.

13. The method of claim 8 wherein the cancer is colon cancer.

14. A kit for diagnosis and prognosis of cancer in a subject comprising a component for detecting the presence S100 protein in a biological sample.

15. The kit of claim 14 wherein the component for detecting S100 protein is an anti-S100 antibody.

16. The kit of claim 15 wherein the anti-S100 antibody is labeled.

17. The kit of claim 16 wherein the label is a radioactive, fluorescent, calorimetric or enzyme label.

18. The kit of claim 15 further comprising a labeled second antibody that immunospecifically binds to the anti-S100 antibody.

19. A kit for diagnosis and prognosis of cancer in a subject comprising a component for detecting the presence of S100 autoantibodies in a sample.

20. The kit of claim 19 wherein the component is an S100 antigen.

21. The kit of claim 20 wherein the S100 antigen is labeled.

22. The kit of claim 20 wherein the S100 antigen is linked to a solid phase.

23. The kit of claim 19 further comprising a component for detection of the S100 auto antibody.

24. A method of immunizing a host against an S100 protein, S100 derived peptide or differentially modified S100 protein, comprising inoculating the host with an S100 antigen in a physiologically acceptable carrier, wherein immunization results in a production of antibodies directed against said S100 antigen.

25. The method of claim 24 wherein the host is suffering from a disease characterized by the overproduction of S100 protein.

26. The method of claim 25 wherein the disease is cancer.

27. The method of claim 26 wherein the cancer is lung cancer.

28. The method of claim 26 wherein the cancer is breast cancer.

29. The method of claim 26 wherein the cancer is colon cancer.

30. The method of claim 24 wherein the S100 protein is selected from the group consisting of S100-AG, S100-A7, S100-A8 and S100-A9.

31. A composition for immunizing a host comprising at least one S100 protein and an adjuvant.

32. The composition of claim 31 wherein the S100 protein is selected from the group consisting of S100-AG, S100-A7, S100-A8 and S100-A9.
